(19) United States
(12) Patent Application Publication
(10) Pub. No.: US 2023/0070316 A1
(43) Pub. Date: Mar. 9, 2023

(54) SELF-SENSING CANTILEVER-BASED DEVICES FOR DETERMINING CORNEAL BIOMECHANICS

(71) Applicants: The University of North Carolina at Chapel Hill, Chapel Hill, NC (US); UNIVERSITY OF MIAMI COLLEGE OF ENGINEERING, Coral Gables, FL (US)

(72) Inventors: O'Rese J. Knight, Chapel Hill, NC (US); Devin K. Hubbard, Chapel Hill, NC (US); Noel Marysa Ziebarth, Coral Gables, FL (US)

(21) Appl. No.: 17/799,100

(22) PCT Filed: Feb. 12, 2021
(86) PCT No.: PCT/US21/17919

§ 371 Date: Aug. 11, 2022

Related U.S. Application Data
(60) Provisional application No. 62/975,971, filed on Feb. 13, 2020.

(51) Int. Cl. A61B 3/16; G02C 7/04

(52) U.S. Cl. CPC A61B 3/16; G02C 7/04

(57) ABSTRACT

Devices for determining one or more corneal biomechanical properties are described herein which, in some embodiments, exhibit the versatility for continuous and intermittent patient monitoring. In some embodiments, a device comprises at least one self-sensing cantilever calibrated against a control of known biomechanical properties, wherein the self-sensing cantilever is coupled to a base configured to position the self-sensing cantilever adjacent to a corneal surface.

### RELATED APPLICATION DATA

This application claims priority to pursuant to Article 8 of the Patent Cooperation Treaty to U.S. Provisional Patent Application No. 62/975,971 filed Feb. 13, 2020, the entirety of which is incorporated by reference herein.

### STATEMENT OF GOVERNMENT RIGHTS

This invention was made with government support under Grant Nos. EY026098 and TR001111 awarded by the National Institutes of Health. The government has certain rights in the invention.

### FIELD

The present invention relates to devices for determining one or more corneal biomechanical properties and, in particular, to devices comprising one or more self-sensing cantilevers for probing corneal surfaces.

### BACKGROUND

Glaucoma is a serious and complex eye disease that can induce optic nerve damage and visual field loss. Glaucoma is generally linked to high intraocular pressure (IOP). Accordingly, IOP is routinely measured in eye exams as a tool for the screening, diagnosis and management of glaucoma. Many ophthalmologists measure IOP with the Goldman applanation tonometer. This device makes the incorrect assumption that the cornea is a thin membrane. Moreover, the applanation tonometer acquires a single IOP measurement during the eye exam. IOP varies throughout the day with a baseline circadian rhythm and in response to physical activity, recumbency, and the cardiac cycle. Therefore, a single measurement fails to provide a complete picture of one or more eye indications.

### SUMMARY

In view of these disadvantages, devices for determining one or more corneal biomechanical properties are described herein which, in some embodiments, exhibit the versatility for continuous and intermittent IOP monitoring. In some embodiments, a device comprises at least one self-sensing cantilever calibrated against a control of known biomechanical properties, wherein the self-sensing cantilever is coupled to a base configured to position the self-sensing cantilever adjacent to or in contact with a corneal surface. In some embodiments, for example, the base can be coupled to a prism of an applanation tonometer. Alternatively, the base can be coupled to a lens, such as a contact lens.

In another aspect, methods of determining one or more corneal biomechanical properties are described herein. A method, in some embodiments, comprises providing a device including at least one self-sensing cantilever calibrated against a control of known biomechanical properties, and positioning the self-sensing cantilever adjacent to or in contact with a corneal surface. The corneal surface is probed with the self-sensing cantilever, and a value is assigned to the one or more corneal biomechanical properties based on output signal of the self-sensing cantilever.

In a further aspect, methods of determining IOP of a patient are described herein. In some embodiments, a method comprises providing a device including at least one self-sensing cantilever calibrated against a control of known biomechanical properties, and positioning the self-sensing cantilever adjacent to a corneal surface of the patient. The corneal surface of the patient is probed with the self-sensing cantilever, and a value is assigned to the corneal Young's modulus of the patient based on output signal of the self-sensing cantilever. IOP is subsequently derived from the value of the patient's corneal Young's modulus.

These and other embodiments are further described in the following detailed description.

### BRIEF DESCRIPTION OF THE DRAWINGS

FIG. 1 illustrates application of a device described herein to a prism of an applanation tonometer according to some embodiments;

FIG. 2 illustrates association of devices described herein with a lens according to some embodiments;

FIG. 3 shows a diagram of an example atomic force microscopy (AFM) system, in accordance with some aspects of the technology described herein;

FIG. 4 illustrates example measurements of properties of an artificial cornea, in accordance with some aspects of the technology described herein;

FIGS. 5A-5B show example self-sensing cantilevers and incorporation in an example AFM system, in accordance with some aspects of the technology described herein;

FIGS. 6A-6B illustrate example measurements using a self-sensing cantilever, in accordance with some aspects of the technology described herein;

FIGS. 7A-7D illustrate example measurements of properties of an artificial cornea, in accordance with some aspects of the technology described herein;

FIGS. 8A-8D illustrate example measurements of properties of an artificial cornea, in accordance with some aspects of the technology described herein;

FIG. 9 illustrates example measurements of properties of an artificial cornea, in accordance with some aspects of the technology described herein; and

FIGS. 10A-10B illustrate example measurements of properties of an artificial cornea, in accordance with some aspects of the technology described herein.

### DETAILED DESCRIPTION

Embodiments described herein can be understood more readily by reference to the following detailed description, examples, and drawings and their previous and following descriptions. Elements, apparatus and methods described herein, however, are not limited to the specific embodiments presented in the detailed description, examples and drawings. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “from 5 to 10” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

### I. Self-Sensing Cantilever Devices

In one aspect, a device described herein comprises at least one self-sensing cantilever calibrated against a control of known biomechanical properties, wherein the self-sensing cantilever is coupled to a base configured to position the self-sensing cantilever adjacent to a corneal surface. In some embodiments, the device comprises a plurality of self-sensing cantilevers. Turning now to specific components, any self-sensing cantilever not inconsistent with the technical objectives described herein can be employed. The self-sensing cantilever, for example, can comprise any tip geometry for probing corneal surfaces. In some embodiments, the tip exhibits a rounded geometry with radius of curvature. The radius of curvature can have any desired value. In some embodiments, for example, the radius of curvature is from 1 μm to 50 μm. Moreover, the self-sensing cantilever, in some embodiments, can comprise piezo-resistive circuitry for signal generation in response to cantilever deflection. The self-sensing cantilever can be associated with a resonant circuit having frequency varying with respect to resistance of the cantilever tip. A resonant stimulator for activation of the self-sensing cantilever can also be employed. The resonant stimulator, for example, can pulse an electromagnetic signal to activate circuitry of the self-sensing cantilever.

Electrical apparatus associated with the self-sensing cantilever can also include a receiving circuit for receiving signal(s) from the self-sensing cantilever. Signal output may be routed through an amplifier, in some embodiments. Additional components can include analog-to-digital converter, digital storage, digital signal processing, signal filtration apparatus, and/or environmental compensation circuitry to address signal variations resulting from factors including temperature, orientation, motion, and/or other data received from auxiliary components. Electrical components associated with the self-sensing cantilever can be powered by one or more battery packs and/or other power sources including photovoltaic, thermoelectric and/or tribological power sources.

As described herein, the self-sensing cantilever is calibrated against a control of known biomechanical properties. In some embodiments, the control is a test eye, such as that obtained from an animal. A test eye, for example, may be a porcine test eye, in some embodiments. Alternatively, the control may be a synthetic sample mimicking corneal biomechanical properties. Calibration of the self-sensing cantilever against the known control enables output signal/data of the self-sensing cantilever to be assigned or correlated to accurate values for various corneal biomechanical properties, such as Young's modulus of elasticity of the full thickness human cornea. In some embodiments, the self-sensing cantilever is placed into a standard atomic force microscopy (AFM) set up and calibrated using traditional, split diode arrangement with a laser reflected surface. Displacements and force outputs can be correlated to the self-sensing cantilever output.

The self-sensing cantilever can be coupled to a base, the base configured to position the self-sensing cantilever adjacent to the corneal surface. Any base not inconsistent with the technical objectives described herein can be employed. In some embodiments, the base is a ring or annular base. The self-sensing cantilever can have any desired orientation relative to the base. In some embodiments, the cantilever tip faces inward from the ring perimeter. Alternatively, the cantilever tip may face outward from the ring perimeter. Moreover, the self-sensing cantilever may lie in the same plane as the ring or may be inclined relative to the ring. In some embodiments, the cantilever is straight or linear. Alternatively, the cantilever may exhibit curvature. The curvature may be continuous along the cantilever or the curvature may be interrupted by one or more linear sections. The cantilever, for example, may exhibit curvature addressing one or more contours of the cantilever environment, such as the contour of a contact lens and/or contour of the ocular environment. When multiple self-sensing cantilevers are present on the device, the cantilevers may exhibit similar orientations relative to the base. In other embodiments, the cantilevers may have differing orientations relative to the base.

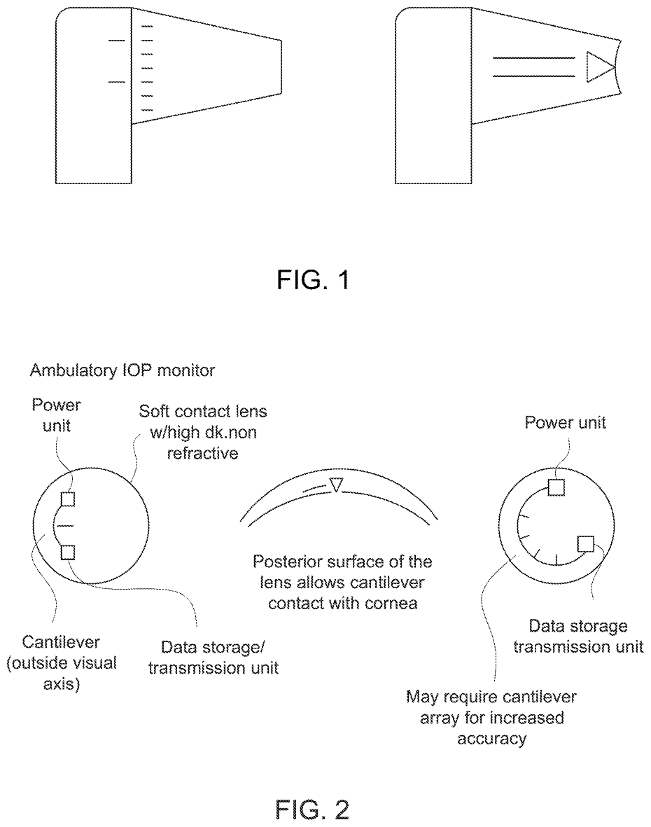

In some embodiments, devices described herein can be coupled to a prism of an applanation tonometer. In such embodiments, the device can be employed to determine one or more corneal biomechanical properties during an eye exam of the patient. In some embodiments, the prism can comprise a contoured surface for assessing corneal curvature while the device comprising the self-sensing cantilever determines corneal Young's modulus. As described further herein, these parameters can be used to derive IOP of the patient. FIG. 1 illustrates one embodiment of application of the device to a prism of an applanation tonometer.

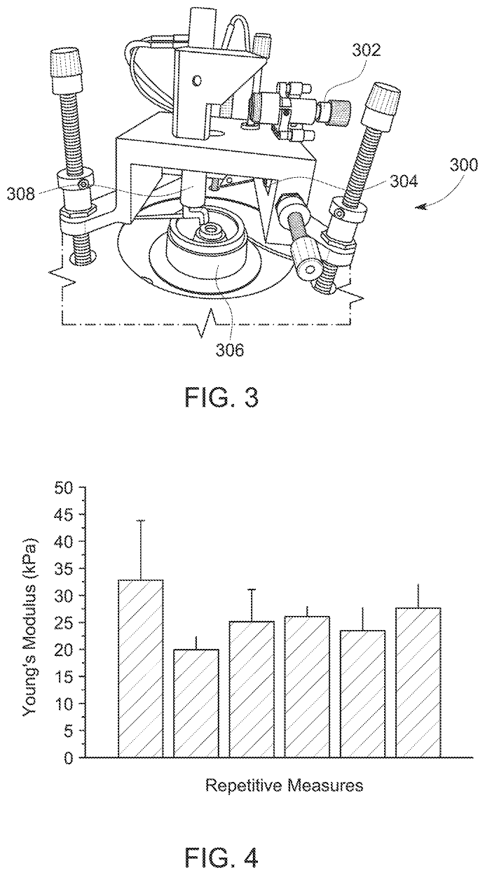

In another embodiment, the device can be coupled to a lens, such as a contact lens or a flexible corneal lens. The base, for example, can be coupled to, or encapsulated in, the contact lens material for positioning the one or more self-sensing cantilevers adjacent to the corneal surface. When coupled to a contact lens or other lens, devices described herein can be used for continuous measuring or monitoring of one or more corneal biomechanical properties. Such data can be employed in developing a detailed and accurate assessment of eye health. In some embodiments, the device may comprise wireless data transmission apparatus for passing data to one or more electronic monitoring devices, such as a mobile phone or computer. FIG. 2 illustrates the association of devices described herein with a lens according to some embodiments. FIG. 2, for example, illustrates one embodiment wherein a device comprising a single self-sensing cantilever is coupled to a contact lens. Another embodiment is also illustrated wherein the device comprises an array of cantilevers. In some embodiments, an array of cantilevers permits measurement of corneal biomechanical properties at multiple surface locations, thereby providing additional data points for analysis. A lens comprising a device described herein can further comprise a skirt or other feature for contacting the limbus or sclera of the eye to aid in proper positioning of the lens. In some embodiments, the skirt may support or house one or more components of the self-sensing cantilever device including, but not limited, to circuitry, sensor(s) power component(s), and/or data management components.

While self-sensing cantilevers are described herein, the foregoing embodiments can be achieved with other types of cantilevers. Accordingly, embodiments described herein are not limited to the use of self-sensing cantilevers. Any cantilever operable to achieve the technical objectives described herein can be employed.

### II. Methods of Determining Corneal Biomechanical Properties

In another aspect, methods of determining one or more corneal biomechanical properties are described herein. A method, in some embodiments, comprises providing a device including at least one self-sensing cantilever calibrated against a control of known biomechanical properties, and positioning the self-sensing cantilever adjacent to or in contact with a corneal surface. The corneal surface is probed with the self-sensing cantilever, and a value is assigned to the one or more corneal biomechanical properties based on output signal of the self-sensing cantilever.

In a further aspect, methods of determining intraocular pressure (IOP) of a patient are described herein. In some embodiments, a method comprises providing a device including at least one self-sensing cantilever calibrated against a control of known biomechanical properties, and positioning the self-sensing cantilever adjacent to a corneal surface of the patient. The corneal surface of the patient is probed with the self-sensing cantilever, and a value is assigned to the corneal Young's modulus of the patient based on the output signal of the self-sensing cantilever. TOP is subsequently derived from the value of the patient's corneal Young's modulus.

In some embodiments, the Young's modulus is combined with corneal radius of curvature and/or central corneal thickness to derive patient TOP. Devices employed in methods described herein can have any design, architecture, calibration, and/or properties detailed in Section I hereinabove.

In one non-limiting embodiment, TOP can be derived from a patient's corneal Young's modulus according to the following non-limiting analysis. The self-sensing cantilever can be treated as a spherical indenter, in some embodiments. The Hertz model for a spherical indenter is:

$$F = \frac{4 E \sqrt{R}}{3 ( 1 - {v}^{2} )} {D}^{\frac{3}{2}}$$

where F is the measured force, R is the radius of the spherical indenter, and D is the measured indentation. The resultant equation describing the relationship between TOP and the biomechanical properties of the cornea is:

$$E = \frac{1}{7500} ( \frac{3 F ( 1 - {υ}^{2} )}{4 \sqrt{{R}_{AFM}}} {D}^{- \frac{3}{2}} - \frac{{{R}_{C} ( {R}_{C} - 0.5 * CCT )}^{2} ( 1 - v )}{{R}_{AFM}^{2} * CCT} IOPT )$$

where R_{AFM} is the radius of curvature of the self-sensing cantilever tip. R_{C} and CCT are corneal radius of curvature and central corneal thickness, respectively. R_{C} and CCT can vary considerably between patients and in the case of prolonged wear of contact lenses. These will be measured separately during the experiment to develop the empirical relationship. Describing the empirical relationship will provide a set of expected values over a range of TOP, CCT, R_{C}, and E. The foregoing equations are examples of deriving TOP from measured corneal Young's modulus. Additional analytical techniques correlating corneal Young's modulus to TOP are also contemplated.

### III. EXAMPLES

Embodiments described herein can be understood more readily by reference to the following Examples. Elements, apparatus, and methods described herein, however, are not limited to any specific embodiment presented in the Examples. It should be recognized that these are merely illustrative of some principles of this disclosure, and are non-limiting. Numerous modifications and adaptations will be readily apparent without departing from the spirit and the scope of this disclosure.

As described herein, the calibration of intraocular pressure assessment instruments, such as a self-sensing cantilever, control enables output signal/data of the self-sensing cantilever to be assigned or correlated to accurate values for various corneal biomechanical properties, such as Young's modulus of elasticity of the full thickness human cornea. It will be appreciated, that traditional methods of measuring intraocular pressure are not correctly calibrated for accurately measuring various corneal biomechanical properties, such as a corneal Young's modulus of elasticity. Accordingly, in order to determine a true intraocular pressure as described herein, assessment instruments can first be normalized for a given patient's corneal modulus of elasticity.

As will be appreciated through the following examples and experimental procedures, atomic force microscopy (AFM) can be leveraged to directly measure a corneal modulus of elasticity over a physiological range of intraocular pressure. Utilizing the corneal modulus of elasticity measurements, a relationship between intraocular pressure and the modulus of elasticity can be determined. Accordingly, devices and methods described herein may demonstrate sensitivity to changes in intraocular pressure based on surface measurements of corneal modulus of elasticity.

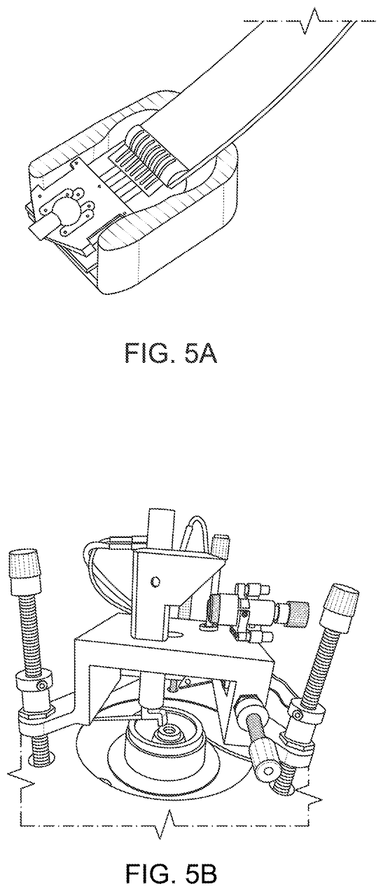

Looking initially at FIG. 3, a custom AFM setup 300 for quantifying Young's modulus of elasticity on a sample is provided. An AFM can measure Young's modulus of elasticity via the indentation of a sample surface using a cantilever. A custom AFM setup 300 can have a variety of components, for example, among other components, custom AFM setup 300 can include a diode laser 302, a position sensitive photodiode 304, and a cantilever holder 306. Piezoelectric mechanism 308 is used to lower a cantilever and indent a sample. In response the cantilever can deflect an amount dependent on the softness of the sample, i.e. the harder the sample, the more the cantilever will deflect. The deflection of the cantilever is proportional to the force that an AFM probe tip exerts on a given sample, thus recorded cantilever deflection-indentation curves can be used to derive force-indentation curves for a measured sample when the deflection response of the cantilever measuring the sample is known, for instance the deflection response of the cantilever on a hard surface. As described herein, the custom AFM setup 300 can further include a piezoresistive self-sensing cantilever, that may further incorporate driving electronics and a housing. As an output of a self-sensing cantilever coupled to an AFM is a voltage, calibration is necessary to bring it into alignment with a traditional AFM setup.

### 1. AFM Measurement of Biomechanical Properties of Artificial Corneas at 0 mm Hg

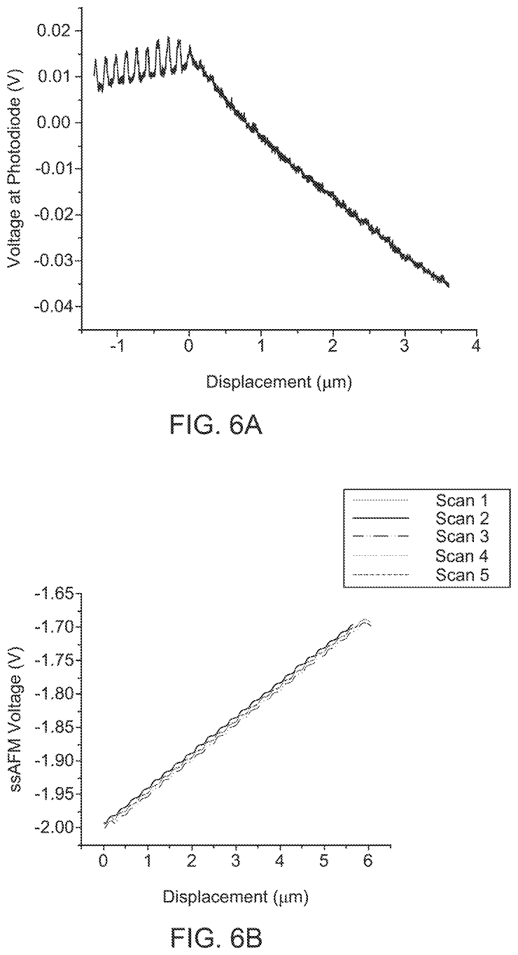

As described herein, the self-sensing cantilever is calibrated against a control of known biomechanical properties, for example the self-sensing cantilever is placed into a standard atomic force microscopy (AFM) set up and calibrated using traditional, split diode arrangement with a laser reflected surface. Displacements and force outputs can be correlated to the self-sensing cantilever output. This instant example illustrates AFM measurements of biomechanical properties of an artificial cornea at 0 mmHg, as such the control is a synthetic sample that mimics corneal biomechanical properties. Artificial corneal buttons were provided and were adhered to a standard 35 mm Petri dish. Each Petri dish having an adhered corneal button were then filled with warm, ˜75-95° F., deionized water until the artificial cornea was covered. The submerged corneal button (artificial cornea) was then allowed to equilibrate for about 10 minutes. Using an AFM setup (e.g. AFM setup 300 of FIG. 3). Elasticity scans were subsequently performed at different locations around the central region of the corneal buttons. These scans were then analyzed to calculate Young's modulus of elasticity for the artificial cornea, and the scans and modulus of elasticity calculations were repeated over three different days to ensure consistency. Calibration results obtained over three separate days of measurements, as illustrated in Table 1 below, showed artificial corneas under the measured conditions had a Young's modulus of elasticity of around 25.8±4.3 kPa. Additionally, FIG. 4 graphically illustrates the described measurements and calculation of Young's modulus of elasticity for the artificial corneal buttons, where each column represents a series of independent measurements taken.

TABLE 1

| Day of | | Young's Modulus |
|---|---|---|
| Measurement | Position | (kPa) |
| 1 | 1 | 32.8 ± 11.1 |
| | 2 | 19.9 ± 2.4 |
| 2 | 1 | 25.2 ± 5.9 |
| | 2 | 26.0 ± 1.9 |
| 3 | 1 | 23.5 ± 4.3 |
| | 2 | 27.6 ± 4.3 |

2. AFM Measurement of Biomechanical Properties Using ssAFM

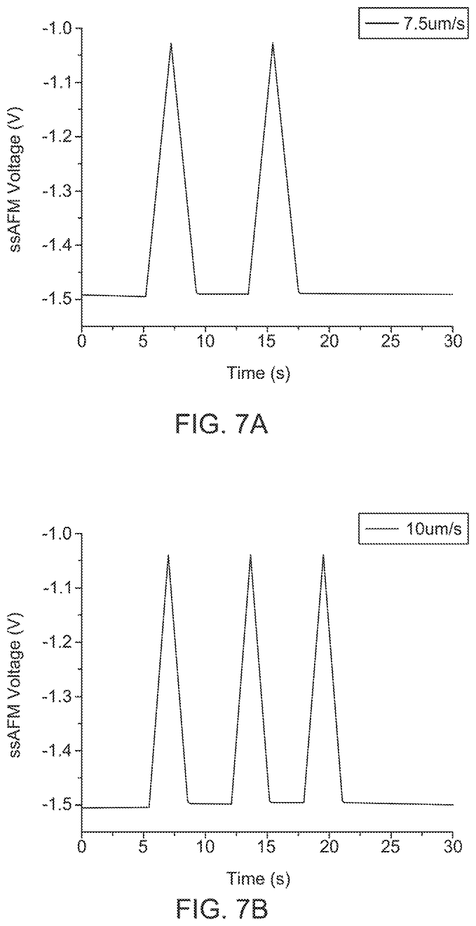

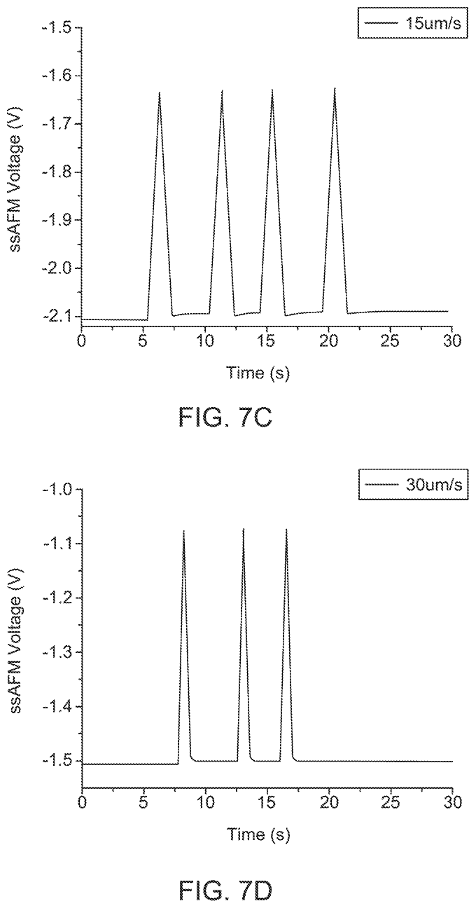

A custom holder was developed to allow the ssAFM cantilever to be connected directly to the piezoelectric mechanism used in a standard or custom AFM system. As depicted in FIGS. 5A and 5B, an ssAFM cantilever (i.e. self-sensing cantilever coupled to an AFM system) can be connected to a traditional or custom AFM setup (e.g. AFM setup 300 of FIG. 3). Traditional AFM scans measuring the deflection-indentation relationship of a hard Petri dish were conducted using the ssAFM implemented in the AFM setup as the probe. As shown in FIGS. 6A and 6B, scans on a petri dish with the ssAFM probe were read simultaneously with a recordation of the voltage output of the ssAFM. The slopes of the output from both traditional AFM and ssAFM measurements were calculated. The slope from traditional AFM scans was 7.522×10⁻⁵±6.835×10⁻⁷ V/m and the slope from the ssAFM output was 0.0527±0.0002 V/m.

3. ssAFM Measurement of Biomechanical Properties of Artificial Corneas at 0 mmHg

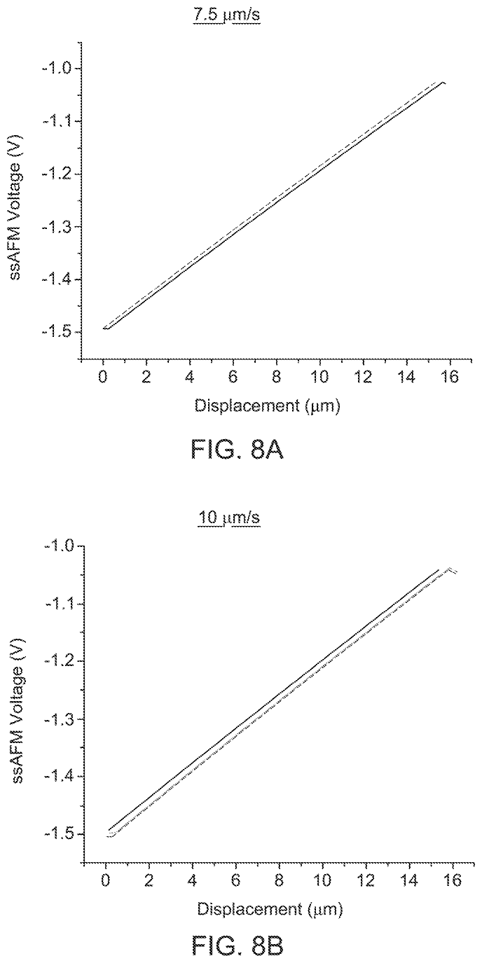

The same corneal buttons used in Example 1 above, were then used to look at ssAFM (as opposed to traditional AFM) measurements of biomechanical properties of artificial corneas. The ssAFM was mounted on the piezoelectric actuator of the custom AFM (e.g. AFM setup 300 of FIG. 3), as shown in FIG. 5B. The piezoelectric actuator was used to indent the cornea 15 μm, while recording the output signal from the ssAFM. The measurements were repeated at four different speeds: 7.5 μm/s, 10 μm/s, 15 μm/s, and 30 μm/s. The ssAFM voltage output as a function of time is shown in FIGS. 7A-7D for the four piezoelectric actuator speeds used herein. Looking at FIGS. 7A-7D each signal peak represents one indentation and one retraction of the piezoelectric actuator from the sample.

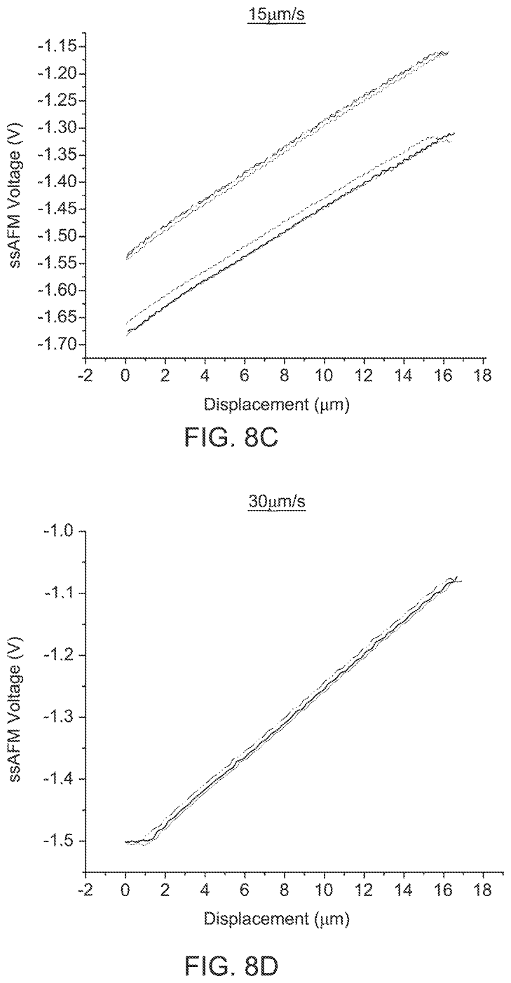

Subsequently, the voltage versus time graphs corresponding to FIGS. 7A-7D were converted into ssAFM voltage versus displacement graphs, illustrated by FIGS. 8A-8D, by incorporating the speed of the piezoelectric actuator, i.e. only the indentation portion of the scans were included. As shown in FIGS. 8A-8D, each indentation consistently corresponded to 15 μm of indentation, which was the setpoint on the piezoelectric actuator.

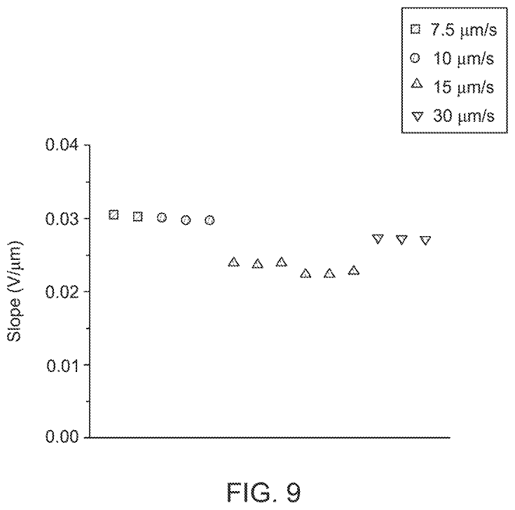

From the graphs of FIGS. 8A-8D, the slope was calculated. As indicated in FIG. 9, the calculated slope was 0.027±0.003 V/μm. The slope can be directly correlated to the sample stiffness, which was found to be approximately 26 kPa using traditional AFM (see Example 1 above). In Example 2 also above, the slope on a hard surface was 0.0527 V/μm, which is approximately double what we find on a much softer artificial cornea. This validates that the slope of the output of the ssAFM is directly proportional to stiffness (i.e. increased slope for stiffer samples). Accordingly, FIG. 9 illustrates the change in ssAFM voltage and slope for the indentations performed at different speeds, and as can be seen both quantities are consistent between the measurements.

4. ssAFM Measurement of Biomechanical Properties of Artificial Corneas Under Pressure

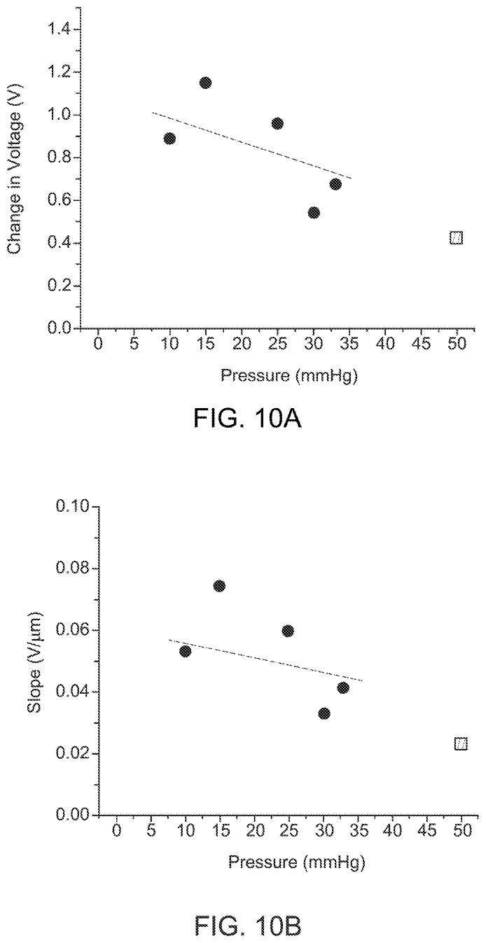

A realistic corneal model designed to practice corneal surgery was provided. The material of the cornea was the same as the corneal buttons that were used in Examples 1 and 3 above. The corneal model was placed in a custom developed pressure chamber that could, for example, mimic intraocular pressure. A pressure sensor of the pressure chamber had a computer-controlled readout, so exact pressure within the chamber could be recorded. The ssAFM was placed in contact with the cornea, and the piezoelectric actuator was used to indent the cornea 15 μm, while recording the output signal from the ssAFM. This was repeated for pressures of 10 mmHg, 15 mmHg, 25 mmHg, 30 mmHg, and 33 mmHg. As illustrated by FIG. 10A, the change in voltage as a function of pressure was recorded, as pressure was increased the output voltage from the ssAFM was recorded. As illustrated by FIG. 10B, the slope of voltage vs. displacement (v/mm) as a function of pressure was determined, as was similarly done in Examples 2 and 3. As can be seen, there was a linear relationship between the change in voltage and the slope recorded by the ssAFM and pressure within the pressure chamber and both have decreasing trends with increasing pressure. As such, these results demonstrate that the ssAFM will export a result directly correlated to pressure. It will be appreciated that the plot point at the highest pressure in FIG. 10A and FIG. 10B shows prior results recorded with a hard surface behind the cornea sample, and thus can approximate an “infinite” pressure case.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A device for determining one or more corneal biomechanical properties comprising:
at least one self-sensing cantilever calibrated against a control of known biomechanical properties, wherein the self-sensing cantilever is coupled to a base configured to position the self-sensing cantilever adjacent to or in contact with a corneal surface.

2. The device of claim 1, wherein the self-sensing cantilever comprises piezo-resistive electrical circuitry for determining cantilever deflection.

3. The device of claim 1, wherein the base is a ring.

4. The device of claim 3, wherein the cantilever tip faces inward from the ring perimeter.

5. The device of claim 3, wherein the cantilever tip faces outward from the ring perimeter.

6. The device of claim 1, wherein the self-sensing cantilever is coupled to the base at an incline.

7. The device of claim 1 further comprising a power source coupled to the base.

8. The device of claim 1 further comprising a data storage and/or data transmission components.

9. The device of claim 1 comprising a plurality of self-sensing cantilevers.

10. The device of claim 1, wherein the base is coupled to a prism of an applanation tonometer.

11. The device of claim 10, wherein the prism comprises a contoured surface for assessing curvature of the corneal surface.

12. The device of claim 1, wherein the base is coupled to a lens.

13. The device of claim 12, wherein the lens is a contact lens or corneal lens.

14. The device of claim 1, wherein the control comprises corneal tissue.

15. The device of claim 1, wherein the control is a synthetic cornea.

16. The device of claim 1, wherein the one or more biomechanical properties include corneal Young's modulus.

17. The device of claim 1, wherein the self-sensing cantilever has a tip radius less than 50 nm.

18. A method for determining one or more corneal biomechanical properties comprising:
providing a device including at least one self-sensing cantilever calibrated against a control of known biomechanical properties;
positioning the self-sensing cantilever adjacent to or in contact with a corneal surface;
probing the corneal surface with the self-sensing cantilever; and
assigning a value to the one or more corneal biomechanical properties based on output signal of the self-sensing cantilever.

19. The method of claim 18, wherein the one or more biomechanical properties include corneal Young's modulus.

20. The method of claim 18, wherein the self-sensing cantilever is coupled to an annular base.

21. The method of claim 18, wherein the device comprises a plurality of self-sensing cantilevers for probing the corneal surface at multiple locations.

22. The method of claim 18, wherein a tip of the cantilever has a radius of curvature less than 50 nm.

23. The method of claim 18, wherein the device further comprises data storage and/or data transmission components.

24. The method of claim 18, wherein the device is coupled to a prism of an applanation tonometer.

25. The method of claim 24, wherein the prism comprises a contoured surface for assessing curvature of the corneal surface.

26. The method of claim 18, wherein the device is coupled to a posterior surface of a contact lens.

27. The method of claim 26, wherein the probing occurs over at least a 12-hour period.

28. A method of determining intraocular pressure of a patient comprising:
providing a device including at least one self-sensing cantilever calibrated against a control of known biomechanical properties;
positioning the self-sensing cantilever adjacent to or in contact with a corneal surface of the patient;
probing the corneal surface with the self-sensing cantilever;
determining a value of corneal Young's modulus of the patient based on output signal of the self-sensing cantilever; and
deriving the intraocular pressure from the corneal Young's modulus.

29. The method of claim 28 further comprises determining corneal radius of curvature and central corneal thickness.
